(19) United States
(12) Patent Application Publication
(10) Pub. No.: US 2004/0049233 A1
(43) Pub. Date: Mar. 11, 2004

(54) Medical device status information system

(76) Inventor: D. Craig Edwards, Fall City, WA (US)

Correspondence Address: SHUMAKER & SIEFFERT, P. A., 8425 SEASONS PARKWAY, SUITE 105, ST. PAUL, MN, 55125, US

(21) Appl. No.: 10/378,001

(22) Filed: Feb. 28, 2003

Related U.S. Application Data
(60) Provisional application No. 60/409,734, filed on Sep. 11, 2002.

(51) Int. Cl. A61N 1/39

(52) U.S. Cl. 607/5

(57) ABSTRACT

In general, the invention is directed to management of status information from a plurality of emergency medical devices, such as AEDs. A system may include one or more medical devices associated with one or more docking stations. The medical devices and docking stations may communicate with one another and with a remote unit. A medical device or a docking station in the system may acquire status information and may communicate the status information to the remote unit, which may serve as a central point for collecting and aggregating status information pertaining to medical devices and docking stations in the system. The remote unit may present the status information to a person via an input/output device, may maintain a status log for the devices in the system, and may interrogate the devices in the system for status information.

This application claims priority from U.S. Provisional Application Serial No. 60/409,734, filed Sep. 11, 2002, the entire content of which is incorporated herein by reference.

### TECHNICAL FIELD

The invention relates to medical devices, and in particular, to collection of status data generated by an emergency medical device or associated apparatus.

### BACKGROUND

Cardiac arrest is a life-threatening medical condition that may be treated with external defibrillation. External defibrillation includes applying electrodes to the patient's chest and delivering an electric shock to the patient to depolarize the patient's heart and restore normal sinus rhythm. The chance that a patient's heart can be successfully defibrillated increase significantly if a defibrillation pulse is applied quickly.

Until recently, individuals such as paramedics, emergency medical technicians, police officers and others trained in defibrillation techniques used defibrillators, but the general public did not. In some cases, the patient's need is urgent and the patient cannot wait for trained personnel to arrive. In recognition of the need for prompt treatment, automated external defibrillators (AEDs) are becoming more commonplace, and are available in venues such as health clubs and auditoriums. In some large venues, such as office buildings, factories, airports and sports arenas, several AEDs may be deployed throughout the venue. In some venues, hundreds of AEDs may be deployed. Ready availability of AEDs may mean that patients may get needed treatment promptly, and need not wait for emergency personnel to arrive. As a result, more lives may be saved.

As part of ordinary security and maintenance procedures, AEDs deployed in a venue may be periodically checked. A responsible person, such as security personnel, may be assigned to make an inspection of each AED and confirm that the device is operational. The inspection may be relatively simple, because many AEDs perform one or more automatic self-diagnostic routines and provide one or more status indications that the device is operational or in need of service.

As part of the inspection, the responsible person should regularly look at each AED and check the associated status indicators. The responsible person may also be required to prepare and maintain records showing that the inspections have been performed, as well as the status and repair history of the AEDs. In a venue having several AEDs, the cost of inspection may be significant. A deployed AED may be unprepared to provide defibrillation therapy if the responsible person makes an inspection error. In addition, a deployed AED may be unprepared to provide defibrillation therapy if a fault or other problem occurs following an inspection.

Because AEDs may be deployed in venues accessible to the public, AEDs may be prone to mischief or misuse. Theft, inadvertent or inappropriate use, tampering, vandalism and the like may be important concerns. Because of these concerns, AEDs may be deployed with a docking station that deters mischief or misuse. An example of such a docking station is a wall-mounted cabinet with a glass window and an alarm system. The alarm may be triggered when the door of the cabinet is opened or if the AED is removed.

Many of the concerns applicable to AEDs may be applicable to other emergency medical devices as well. For example, there may be benefits associated with deploying medical devices such as a stroke apparatus, a chest compression device, or a first aid device, throughout a venue. These medical devices, like AEDs, may be inspected as part of ordinary security and maintenance procedures. To deter mischief or misuse, the medical devices may be deployed with docking stations.

### SUMMARY

In general, the invention is directed to management of status information from a plurality of emergency medical devices, such as AEDs. A system for managing status information may include one or more medical devices associated with one or more docking stations. A medical device, or a docking station, or both, may acquire status information and may communicate the status information to a remote unit. The remote unit may be a status monitor that receives status information from a plurality of medical devices and docking stations in a system.

The remote unit provides a central point for collecting and aggregating status information pertaining to medical devices and docking stations in the system. The remote unit may present the status information to a person via an input/output device, and may maintain a status log that records the status and repair history of the devices in the system.

The remote unit may, in some embodiments, interrogate one or more medical devices or one or more docking stations in the system. In response to the interrogation, the interrogated device may perform a self-diagnostic routine, thereby acquiring status information about its own operating status. The interrogated device may transmit the status information to the remote unit.

The invention is not limited to systems in which every medical device is associated with a docking station. For practical reasons that will be described in more detail below, however, it may be advantageous for a medical device to be associated with a docking station. It may further be advantageous for the medical device and the docking station to communicate with one another. The communication may involve interrogations for status information, as well as status information itself.

When the medical device and the docking station communicate, the docking station may locally present some of the status information received from the medical device. The docking station may include one or more output elements, such as a visual display or annunciator or a speaker, that presents at least a portion of the status information from the medical device. The docking station may also present at least a portion of the status information about itself. A person wishing to perform a routine visual inspection of a medical device and the associated docking station may readily obtain status information about both devices by looking at the output elements on the docking station.

In one embodiment, the invention is directed to a method that may be practiced by a docking station. The method comprises acquiring first status information from a self-diagnostic routine, acquiring second status information from an emergency medical device and communicating the first status information and the second status information to a remote unit. The method may also include presenting at least a portion of the first status information and the second status information to a person.

In another embodiment, the invention is directed to a method that may be practiced by an emergency medical device. The method comprises receiving an interrogation for an emergency medical device status information, acquiring the status information from a self-diagnostic routine and communicating the status information to a remote unit.

In a further embodiment, the invention is directed to methods that may be practiced by a status monitor. One such method includes receiving emergency medical device status information from a plurality of emergency medical devices and presenting at least a portion of the status information to a person. Another such method includes transmitting an interrogation for status information to a plurality of emergency medical devices and receiving the status information from the medical devices in response to the interrogation.

The invention further includes computer-readable media comprising instructions for causing a programmable processor to carry out the methods described above.

In an additional embodiment, the invention is directed to a device comprising an electrical source to generate a shock to defibrillate a heart, at least two electrodes to deliver the shock to the heart, a processor to perform a self-diagnostic routine and to acquire status information as a function of performing the self-diagnostic routine, and a communication module to receive an interrogation for the status information and to communicate the status information to a second device. The device may be an AED.

In another embodiment, the invention presents device that includes a docking element to retain an emergency medical device, a communication module to receive first status information from the medical device, a processor to perform a self-diagnostic routine and acquire second status information as a function of performing the self-diagnostic routine, and an output to present at least a portion of the first status information and at least a portion of the second status information.

In an added embodiment, the invention presents a device comprising a communication module to receive status information from a plurality of emergency medical devices and an output device to present at least a portion of the received status information to an operator. The communication module may be further configured to transmit an interrogation to one or more of the medical devices.

In a further embodiment, the invention is directed to a system. The system includes a plurality of emergency medical devices and a remote unit that interrogates at least one medical device in the plurality over a communication network and that receives status information in response to the interrogation. The system may further include a docking station sized to receive at least one medical device in the plurality.

The invention may offer one or more advantages. The invention may be practiced with systems of many configurations. Any number of docking stations and medical devices may be tracked and monitored with the invention. The invention may also be practiced with any number of networks, and may in some cases be integrated into an existing network in the venue, such as a security network or a private building maintenance network.

The invention provides easy monitoring of any number of medical devices and docking stations deployed throughout a venue. Numerous features of the invention may allow a person responsible for inspection to readily observe the status of any device. Visual inspections of some medical devices and docking stations may be conducted at a glance. The invention also simplifies record keeping operations, such as maintenance of a status log.

Some embodiments of the invention advantageously allocate energy. In the context of an AED, for example, which needs energy to provide defibrillation therapy, communication functions that require substantial energy expenditure may be assigned to a docking station. In such an arrangement, the docking station may be principally responsible for communicating with the status monitor, removing an energy burden on the AED.

In the event of a problem with any device in the system, the invention facilitates prompt notification of a responsible person. The notification may be made by the status monitor and by the affected devices. In addition, some embodiments of the invention may provide for interrogation of a medical device or a docking station, prompting the device to perform a self-diagnostic routine that may discover a problem not previously observed.

In an emergency, the invention advantageously may utilize status information to assist with handling the emergency. For example, when an operator retrieves a medical device such as an AED from a docking station, the AED or the docking station may immediately communicate that fact to the status monitor. A responsible person may promptly dispatch security or emergency personnel to the general area in the venue in which the personnel may be needed.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a perspective drawing of an automated external defibrillator in a cabinet docking station according to an embodiment of the invention.

FIG. 2 is a perspective drawing of an automated external defibrillator in a bracket docking station according to another embodiment of the invention.

FIG. 3 is a block diagram illustrating a system for managing status information from a plurality of medical devices such as AEDs, according to an embodiment of the invention.

FIG. 4 is a flow diagram illustrating interrogation of a medical device according to an embodiment of the invention.

FIG. 5 is a flow diagram illustrating interrogation of a medical device according to an alternate embodiment of the invention.

FIG. 6 is a flow diagram illustrating techniques for acquiring, transmitting and presenting status information that may be practiced by a docking station according to an embodiment of the invention.

FIG. 7 is a flow diagram illustrating techniques for acquiring, transmitting and presenting status information that may be practiced by a medical device such as an AED according to an embodiment of the invention.

FIG. 8 is a flow diagram illustrating techniques for managing status information that may be practiced by a status monitor according to an embodiment of the invention.

FIG. 9 is a block diagram illustrating variations in the system depicted in FIG. 3, consistent with various embodiments of the invention.

### DETAILED DESCRIPTION

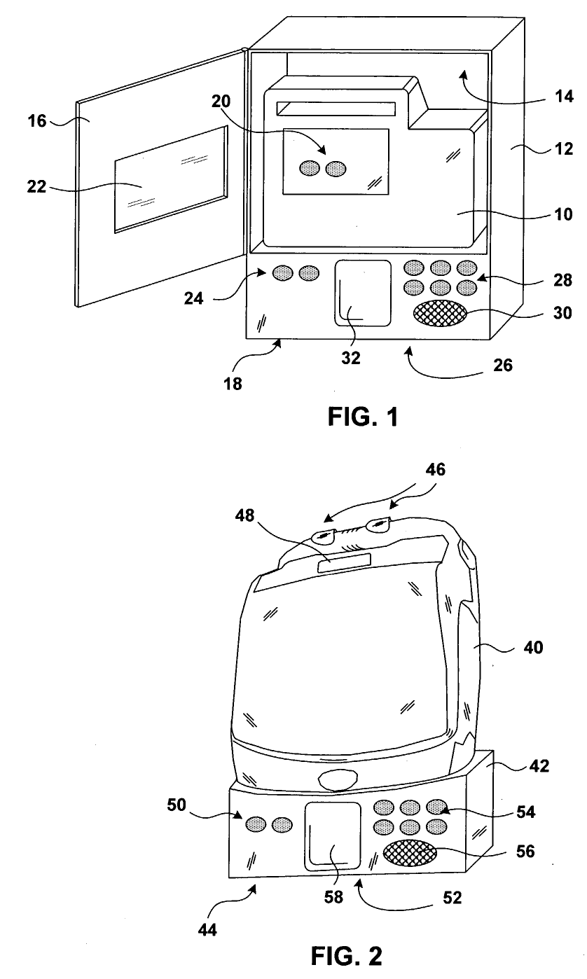

FIG. 1 is a perspective drawing of an automated external defibrillator (AED) 10 in an exemplary docking station 12. AED 10 and docking station 12 are illustrative of the practice of the invention, and for simplicity, the invention will be described in terms of AEDs and docking stations. The invention is not limited to docking stations and AEDs, however, but may include other devices including other types of emergency medical devices.

In the example of FIG. 1, docking station 12 is a cabinet, comprising a compartment 14 that receives AED 10 and a hinged door 16 that closes to secure AED 10 inside compartment 14. AED 10 is portable. When an operator needs to use AED 10, the operator may open door 16 and lift AED 10 from compartment 14. Cabinet 12 also includes a base 18.

AED 10 is capable of administering defibrillation therapy to a patient. AED 10 includes an electrical source (not shown) that can generate one or more shocks to defibrillate the heart of a patient. The shocks may be delivered to the patient via two electrodes (not shown), which may be hand-held electrode paddles or adhesive electrode pads placed externally on the skin of the patient.

The electrodes may be packaged in a sealed pouch (not shown), such as an airtight foil bag, which protects the electrodes from the environment. The electrodes may include substances that may degrade or dry out when exposed to air. For example, the electrodes may include a hydrogel layer that hydrates the patient's skin, forms an interface with the patient, promotes adhesion of the electrodes to the skin and reduces the risk of burns. The electrodes may be stored in a pouch to prevent the hydrogel from drying out and losing its desirable properties. The pouch may be stowed inside AED 10 or inside cabinet 12.

An operator using AED 10 use typically opens the pouch, retrieves the electrodes and places the electrodes in the correct positions on the patient's chest. In some models of AED 10, the operator may also couple the electrodes to AED 10 by plugging an electrical connector into a receptacle on AED 10.

Electrodes of the kind described above are intended for use on one occasion. Following use, the electrodes are discarded, and AED 10 may be supplied with a fresh pouch. Even if the electrodes are not used, however, the electrodes may have a shelf life. The pouch should be replaced when the shelf life expires.

AED 10 may include an internal power source (not shown). The power source for many models of AED 10 is a battery, although some models of AED may be capable of being “line powered,” i.e., plugged into an electrical outlet. Battery power is advantageous in many respects. First, in many situations, the patient may be far from an electrical outlet. In those situations, AED 10 may rely upon a battery to supply the energy for the defibrillation shocks. Second, a power supply in the form of a battery makes AED 10 portable and useful in a wider variety of emergency situations.

AED 10 also comprises an energy storage device (not shown), such as one or more capacitors, and a charging circuit (not shown), such as a flyback charger. When a defibrillation shock is needed, the charging circuit transfers energy from the power supply to the energy storage device. When the energy stored in the energy storage device reaches a desired level, AED 10 is ready to deliver defibrillation therapy. The therapy may be delivered automatically or manually.

AED 10 may further include a microprocessor (not shown) that controls various functions of AED 10. The microprocessor may govern charging of the energy storage device, for example, or may evaluate heart rhythms of the patient sensed via the electrodes, or may deliver the defibrillation shocks automatically. The microprocessor may further execute a routine that performs a self-diagnostic test of AED 10 and acquire status information as a function of performing the self-diagnostic routine.

Status information pertains to the operating status of AED 10 and its attendant components. Status information may include, for example, data indicative of AED 10 being in good working order. Status information may also include data indicative of a fault or potential problem with AED 10, such as data indicative of a failed or damaged component. Data indicating that the battery is low, or that the battery is failing to hold a charge, are additional examples of AED status information. Status information may also include data indicating that the electrodes or other components are nearing the end of their shelf life.

AED 10 may include one or more output elements 20 that convey status information to a person. As shown in FIG. 1, output elements 20 include visual annunciators, such as light-emitting diodes (LEDs) that illuminate or darken to convey status information. Output elements 20 may, for example, indicate whether AED 10 is in good working order, whether the battery is ready, or whether AED 10 needs service. Output elements 20 may include other or additional annunciators, such as a liquid crystal display (LCD), a cathode ray tube (CRT) display, a strobe, or a speaker that is capable of delivering an audible signal or a spoken message.

Hinged door 16 of cabinet 12 includes a window 22. When AED 10 rests in compartment 14 and door 16 is closed, output elements 20 may be visible through window 22. Base 18 of cabinet 12 also includes AED status output elements 24 that may be redundant of output elements 20 on AED 10. In other words, output elements 24 of cabinet 12 may convey the same status information as output elements 20 of AED 11. Output elements 24 may also convey AED status information in a different way than that conveyed by AED 10. Cabinet 12 may, for example, employ a simplified “OK-NOT OK” indicator system, while AED output elements 20 may be more specific about the nature of any problems.

The redundant presentation of status information may be advantageous in several respects. First, instead of facilitating observation of output elements 20 on AED 10, window 22 may impede observation of output elements 20. Because AED 10 may be recessed in compartment 14, for example, output elements 20 may not be visible through window 22 from all angles. Further, window 22 may be cracked or dirty or reflective of light sources that wash out the visual annunciators. Output elements 24 may also be larger or brighter than output elements 20, allowing the status information to be perceived from a greater distance or from a wider angle of view. Thus, a person wishing to perform a routine visual check on the status of AED 10 may obtain status information about AED 10 more readily.

Cabinet 12 presents status information via output elements 24 upon receiving the status information from AED 10. As will be described in more detail below, AED 10 may establish a communication link with cabinet 12, and may communicate status information to cabinet 12. The communication link may be, but need not be, wireless.

In addition to AED status output elements 24, base 18 includes docking station status output elements 26. Docking station status output elements 26 may include visual annunciators 28, a speaker 30 and a display screen 32. Visual annunciators 28 may comprise, for example, LEDs. Display screen 32 may comprise, for example, an LCD or CRT display.

Docking station status output elements 26 convey status information that is not redundant of status information conveyed by AED status output elements 24. The status information conveyed by docking station status output elements 26 may include status information pertaining to AED 10, status information pertaining to cabinet 12, or status information pertaining to other AEDs. As will be described below, AED 10, or cabinet 12, or both, may be part of a networked system of AEDs, and the status information conveyed by docking station status output elements 26 may include status information pertaining to the networked system of AEDs.

Visual annunciators 28 may convey, for example, that cabinet 12 is in good working order, or that the communication interfaces of cabinet 12 are working properly. Speaker 30 may convey, for example, an alarm signaling that door 16 is open or ajar, or verbal instructions concerning use of AED 10 or cabinet 12. Display screen 32 may convey any information in text or visual form, such as a pictorial instruction for opening door 16, or a text warning that AED 10 is out of service, along with directions for finding the nearest AED in the network that is in service.

FIG. 2 is a perspective drawing of another AED 40 in another exemplary docking station 42. In FIG. 2, docking station 42 is a wall-mounted bracket, rather than a cabinet. Bracket 42 includes a shaped base 44 that receives AED 40 and supports AED 40. Bracket 42 also includes and clasps 46, which, in cooperation with base 44, retain AED 40 and secure AED 40 to bracket 42. Clasps 46 may be flexible. When an operator needs to use AED 40, the operator may pull AED 40 from clasps 46 and lift AED 40 out of base 44.

AED 40 may include one or more output elements 48 that convey status information about AED 40, and base 44 may include AED status output elements 50 that may be redundant of output elements 48. As will be described in more detail below, AED 40 may establish a communication link with bracket 42. AED 40 may communicate status information to bracket 42, which bracket 42 may present via output elements 50 on base 44.

Output elements 48 and 50 may be similar to output elements 20 and 24 shown in FIG. 1. Although output elements 48 are not recessed in a compartment or obscured by a window, output elements 48 may be small or difficult to read at a distance. Output elements 50 may be more easily perceived from a greater distance or from a wider angle of view, allowing a person to readily obtain status information about AED 40.

Base 42 includes docking station status output elements 52. Like docking station status output elements 26 shown in FIG. 1, docking station status output elements 52 may include visual annunciators 54, a speaker 56 and a display screen 58.

The embodiments of an AED and a docking station shown in FIGS. 1 and 2 are for purposes of illustration. The invention is not limited to the arrangements depicted. For example, the invention encompasses embodiments in which the docking station output elements are positioned above the AED, or on multiple sides of the AED. The invention encompasses embodiments that include more or fewer output elements than are shown. The invention also encompasses embodiments that include docking elements to retain the AED other than clasps, shaped bases, cabinets and doors. Docking elements may include clamps, lids, covers, trays, shelves, drawers, latches, and the like.

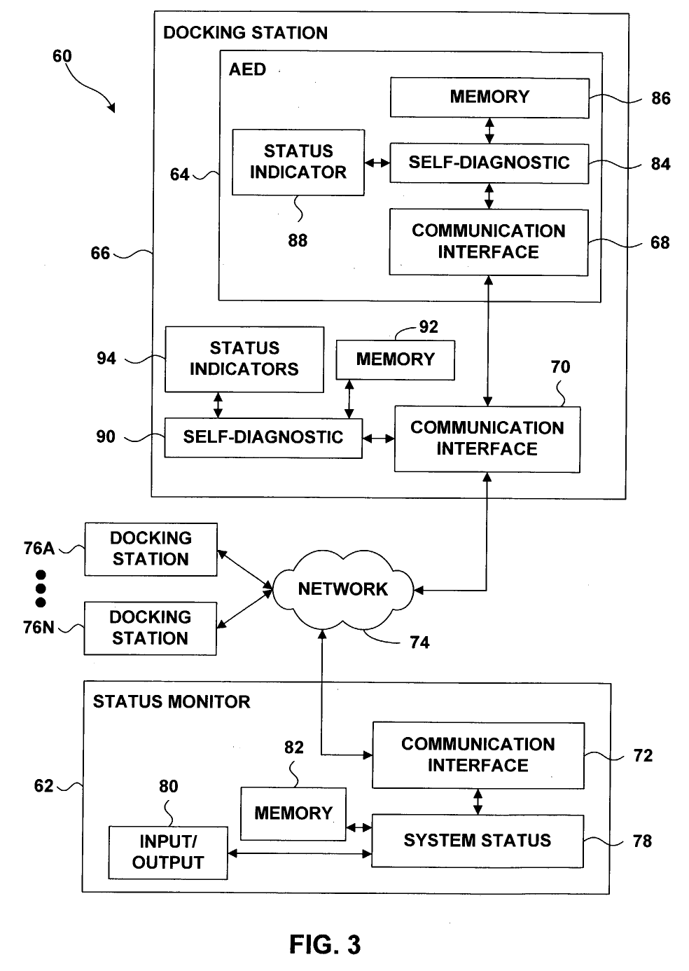

FIG. 3 is a block diagram illustrating an example system 60 in which a status monitor 62 receives status information from an AED 64 and a docking station 66. AED 64 and docking station 66 may be either of the embodiments depicted in FIGS. 1 and 2, but are not limited to those embodiments.

In the embodiment of system 60 shown in FIG. 3, status monitor 62 is a unit that is remote from AED 64 and docking station 66, but is in two-way communication with AED 64 and docking station 66. Status monitor 62 may transmit an interrogation for status information to AED 64 or docking station 66. AED 64 or docking station 66 may perform a self-diagnostic routine to acquire the status information, and may communicate the status information to status monitor 62.

In system 60, AED 64 does not communicate with status monitor 62 directly. Rather, AED 64 communicates with status monitor 62 via docking station 66. In particular, AED 64 includes a communication interface 68 that establishes a communication link with a communication interface 70 in docking station 66. Communication interface 70 in turn establishes a communication link with a communication interface 72 in status monitor 62 over a network 74. Similarly, interrogations from status monitor 62 to AED 64 are communicated through docking station 66.

Network 74 may be any network. Network 74 may comprise, for example, a public switched telephone network, a cellular telephone network, a local area network, a wide area network, a global computer network such as the Internet, an integrated services digital network, or the like. In some venues in which AED 64 and docking station 66 may be deployed, the venue may include a dedicated security network or a private building maintenance network. Either may serve as network 74. Network 74 may include hard-wired electrical or optical communication links, wireless links, or a combination of both.

System 60 is not limited to a single AED 64 or a single docking station 66. Other docking stations 76A-76N may communicate with remote status monitor 62 via network 74. In particular, status monitor 62 may receive status information from docking stations 76A-76N and from AEDs (not shown) associated with docking stations 76A-76N. Status monitor 62 may also transmit interrogations to docking stations 76A-76N and the associated AEDs. AED 64 and docking station 66 are representative of other AEDs and docking stations in system 60.

System status module 78 in status monitor 62 provides a central point for collecting and aggregating status information pertaining to AEDs and docking stations in system 60. In this way, system status module 78 monitors the AEDs and docking stations in system 60. System status module 78 is a processor that may summarize the aggregated status information and present the status information via an input/output device 80. In addition, system status module 78 may interrogate one or more AEDs or one or more docking stations in system 60, and may present to a person status information received in response to the interrogation via input/output device 80. Input/output device 80 may comprise one or more display screens, keyboards, audible alarms, LEDs, LCDs, printers, touch screens, pointing devices, and the like. Input/output device 80 may also comprise a communication device configured to establish a communication link with another person or device not shown in FIG. 3. For example, when status information from AED 64 indicates at problem that may require a professional service call, input/output device 80 may automatically summon the service provider.

System status module 78 may further store information pertaining to the status of system 60, or any AEDs or docking stations in system 60, in memory 82. Information stored in memory 82 may include, for example, routine status information, data pertaining to repair histories, and tracking data showing the locations of devices.

In a typical venue, system status module 78 is remote from AEDs or docking stations in system 60. AEDs and docking stations are ordinarily readily accessible, and in some venues, may be accessible to members of the general public. System status module 78, by contrast, is typically housed in a secure location and is not readily accessible.

In one illustrative embodiment, a personal computer may operate as system status module 78, input/output device 80, and memory 82. In another illustrative embodiment, a portable device such as a pager or personal digital assistant (PDA) may operate as input/output device 80, with system status module 78 and memory 82 located in a different physical location. In this embodiment, system status module 78 and input/output device 80 may communicate via a communication link such as a wireless link or a telephone line. System status module 78 and input/output device 80 may also communicate over network 74.

A responsible person, such as a security supervisor, may observe the status of any AED or docking station in system 60 by observing input/output device 80. Input/output device 80 may notify the responsible person that all AEDs and docking stations in system 60 are operational, for example, or may notify the responsible person when an AED or a docking station in system 60 is in need of attention. When an AED or a docking station in system 60 is in need of attention, input/output device 80 may present the responsible person with information such as the location of the device in question and the nature of the problem. Input/output device 80 may further present the responsible person with status information received from the device in response to an interrogation by system status module 78. Input/output device 80 may also present the responsible person with data stored in memory 82, such as the repair history of the device in question.

AED 64 includes a self-diagnostic module 84 that monitors the status of AED 64. Self-diagnostic module 84 is a processor that executes one or more self-diagnostic routines. The self-diagnostic routines may be initiated by self-diagnostic module 84, or may be initiated in response to a change in the condition of AED 64, such as a component malfunction. By execution of a self-diagnostic routine, self-diagnostic module 84 performs one or more internal self-tests to acquire status information about the state of readiness of AED 64. Self-diagnostic module 84 may evaluate and identify matters that can be customer serviceable, such as battery or electrode replacement, and matters that may require a professional service call. AED 64 may record the status information in memory 86, and may present some or all of the status information via one or more status indicators 88. When the results of the self-tests indicate that AED 64 is ready for use, for example, status indicators 88 may provide a visible or audible indication of readiness. Status indicators 88 may comprise any of output elements 20 or 48 described in connection with FIGS. 1 and 2.

AED 64 may further communicate the status information to docking station 66 via communication interfaces 68 and 70. Communication between AED 64 and docking station 66 may be by an communication technique. In the embodiment shown in FIG. 3, AED 64 and docking station 66 may engage in two-way communication, thereby enabling AED 64 to receive an interrogation from status monitor 62 or docking station 66.

Communication between AED 64 and docking station 66 may be in accordance with one or more wireless communication techniques. For example, one communication protocol, commonly referred to as Bluetooth, uses short-range 2.4 GHz radio technology employed to transport data between devices. Other possible communication protocols include IEEE 802.11a, 802.11b, and 802.11g, which are industry standard protocols for wireless networking. Yet another possible protocol is HomeRF, which was initially designed for wireless communications between devices and appliances within a home.

Communication between AED 64 and docking station 66 may also communicate via a physical communication link. When docking station 66 receives AED 64, mating electrical or optical components in docking station 66 and AED 64 may engage, thereby enabling communication. In addition, AED 64 and docking station 66 may communicate via a combination of wireless and physical communication links. Wireless links and physical communication links both may be implemented so that AED 64 may be quickly and easily removed from docking station 66 without hindrance.

Docking station 66 includes a self-diagnostic module 90 that monitors the status of docking station 66. Self-diagnostic module 90 is a processor that executes a self-diagnostic routine to perform internal self-tests and to acquire status information about docking station 66. The self-diagnostic routines may be initiated by self-diagnostic module 90 or may be initiated in response to a change in the condition of docking station 66. Self-diagnostic module 90 may evaluate and identify matters that can be customer serviceable and matters that may require a professional service call.

In addition, self-diagnostic module 90 may collect, aggregate or interpret status information received from AED 64. In some circumstances, self-diagnostic module 90 may use status information from AED 64 and from self-tests to pinpoint the source of a problem. Self-diagnostic module 90 may record the status information in memory 92, and may present some or all of the status information via one or more status indicators 94. Status indicators 94 may include AED status output elements 24, such as AED status output elements 24 and 50 in FIGS. 1 and 2, that convey AED status information redundantly. Status indicators 94 may also include output elements such as docking station status output elements 26 and 52 in FIGS. 1 and 2. Status indicators 94 may convey status information pertaining to AED 64, status information pertaining to docking station 66, or status information pertaining to other AEDs or docking stations in system 60.

Docking station 66 further includes a power source (not shown in FIG. 3). Unlike AED 64, which is portable and is usually battery-powered, docking station 66 is stationary and may be line-powered. System 60 may include several docking stations 66, 76A-76N deployed throughout a venue, and the docking stations may be wall-mounted or otherwise located for access to the power grid. The invention is not limited to line-powered docking stations, however, but includes docking stations having power sources such as batteries or solar cells.

An advantage of system 60 shown in FIG. 3 is an efficient use of energy. It may be undesirable to devote too much energy from the power supply in AED 64 to communication with docking station 66 or status monitor 62. AED 64 may be battery powered, and the battery power may be needed to supply the energy that may be delivered to a patient as a lifesaving defibrillation shock. Accordingly, the communication resources of AED 64 may be scaled back. When AED 64 is engaged with docking station 66, such as is depicted in FIGS. 1 and 2, AED 64 may not need to expend much energy to communicate with docking station 66.

Docking station 66, by contrast, does not need to conserve energy to provide defibrillation therapy. Moreover, a line-powered docking station 66 may be relieved of the energy constraints that affect a battery-powered AED 64. Accordingly, docking station 66 may devote more energy to communication. In system 60, docking station 66 is responsible for communicating with status monitor 62 and for supplying status monitor 62 with status information about AED 64 and docking station 66. Docking station 66 is also responsible for receiving interrogations from status monitor 62 and relaying interrogations to AED 64.

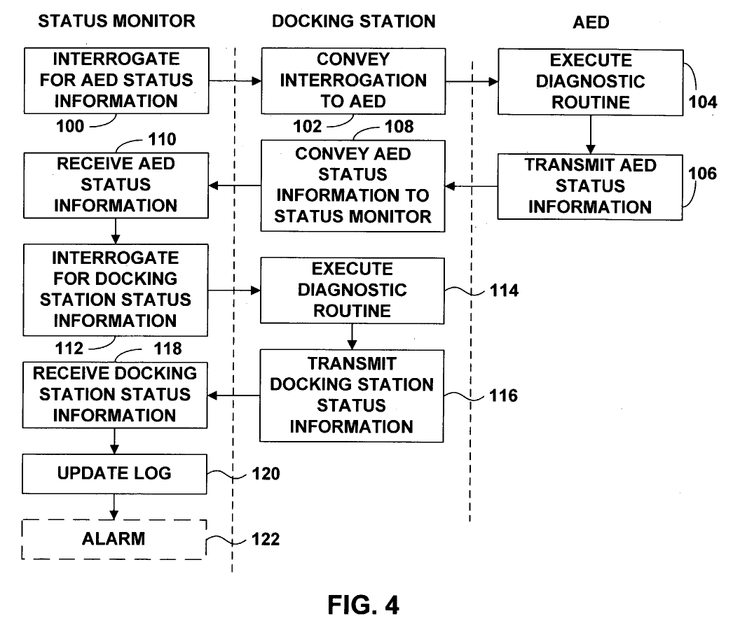

FIG. 4 is a flow diagram illustrating an interrogation in a system such as system 60. Status monitor 62 may interrogate AED 64 by transmitting an interrogation for status information (100). Status monitor 62 may initiate the interrogation in response to a command from a responsible person, or status monitor 62 may initiate the interrogation automatically. An automatic interrogation may be part of a routine periodic interrogation, for example, or the automatic interrogation may be in response to status information received from AED 64, docking station 66 or another device in system 60.

Docking station 66 receives the interrogation and communicates the interrogation to AED 64 (102). In response, the self-diagnostic module 84 of AED 64 executes a self-diagnostic routine to acquire status information about AED 64 (104). AED 64 communicates the status information to docking station 66 (106), which in turn conveys the status information to status monitor 62 (108). Status monitor 62 receives the status information (110).

Status monitor 62 may also interrogate docking station 66 by transmitting an interrogation for status information (112). As with interrogations for AED status information, status monitor 62 may initiate the interrogation in response to a command or automatically. Docking station 66 receives the interrogation executes a self-diagnostic routine to acquire status information about docking station 66 (114). Docking station 66 communicates the status information to status monitor 62 (116). Status monitor 62 receives the status information (118).

When status monitor 62 receives the AED status information and the docking station status information, status monitor may update a status log (120). The status log, which may be stored in memory 82, may include status information pertaining to the readiness of AED 64 and docking station 66. The status log may also record corrective measures that are indicated or that have been taken. When status information received in response to an interrogation indicates a matter requiring prompt attention, status monitor 62 may generate an alarm.(122) to notify the responsible person that corrective action may be required.

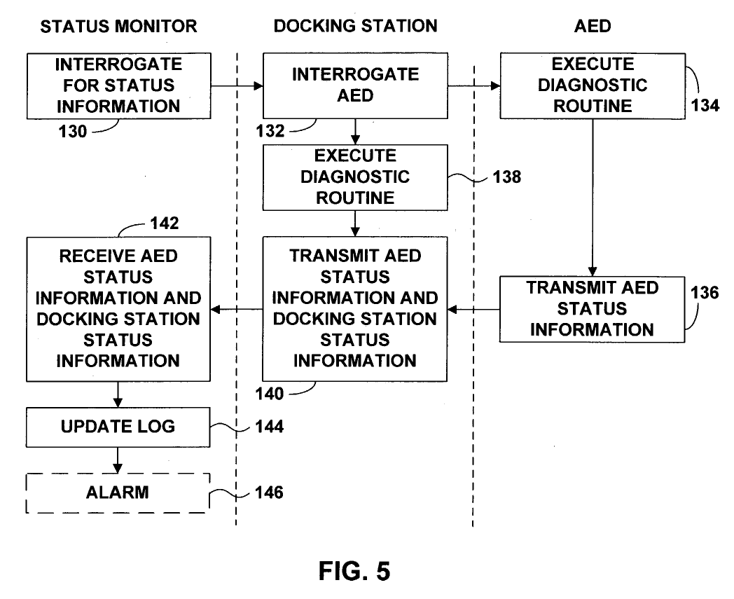

FIG. 5 is a flow diagram illustrating an alternative interrogation technique in a system such as system 60. FIG. 5 is similar to FIG. 4, with a principal difference being that, in FIG. 5, diagnostic operations of AED 64 and docking station 66 are performed in parallel. Status monitor 62 may communicate an interrogation to docking station 66 (130). The interrogation may request AED status information, docking station status information, or status information from both devices.

Upon receipt of the interrogation, docking station 66 generates an interrogation for AED status information, and submits the interrogation to AED 64 (132). In response to the interrogation, AED 64 executes a self-diagnostic routine to acquire AED status information (134), and communicates the status information to docking station 66 (136). In parallel, docking station 66 carries out self-diagnostic tests to acquire docking station status information (138). Docking station 66 communicates the status information to status monitor 62 (140), and status monitor 62 receives the status information (142). Status monitor may update the status log (144) as described above, and may generate an alarm (146) when appropriate.

The techniques depicted in FIGS. 4 and 5 may be performed with several devices in system 60. Status monitor 62 may, for example, submit simultaneous interrogations to all AEDs and docking stations in system 60. Status monitor 62 may also interrogate AEDs and docking stations in system 60 in turn.

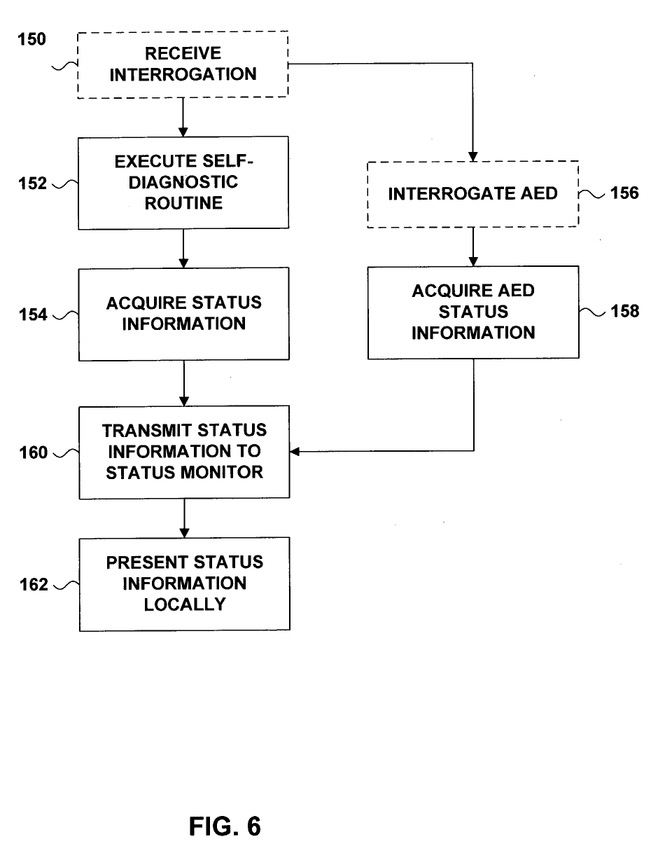

FIG. 6 is a flow diagram illustrating operation of a docking station in a system such as system 60. Upon receipt of an interrogation (150) from a status monitor, the docking station executes a self-diagnostic routine (152) and acquires status information pertaining to the docking station (154). The interrogation (150), however, is optional. The docking station may execute a self-diagnostic routine (152) and acquire status information (154) without an interrogation. The docking station may execute a self-diagnostic routine (152) on a periodic basis, for example, or in response to a detected fault or other problem in the docking station or the AED associated with the docking station.

The docking station may also interrogate the AED associated with the docking station (156), by generating an interrogation for AED status information or by communicating an interrogation to the AED received from a status monitor. The docking station acquires AED status information (158) from the AED. The interrogation from the docking station (156) is optional, and the docking station may acquire AED status information (158) without an interrogation. The docking station may acquire AED status information (158) on a periodic basis, for example, or in response to a detected problem reported by the AED.

The docking station may communicate the status information, which may include docking station status information and AED status information, to a remote unit such as the status monitor (160). The docking station may also present status information locally (162). The docking station may present AED status information via AED status output elements and docking station status information via docking station status output elements.

The docking station may, in addition, receive status information from the status monitor. The status information may be part of an interrogation (150) or may be received in a separate communication. The docking station may present the status information received from the status monitor. For example, the docking station may receive data disclosing the location of the nearest docking station with an operational AED. In the event that the docking station detects a problem with the operational status of itself or its associated AED, the docking station may present the location of the nearest docking station with an operational AED via a display screen. As a result, an operator needing an AED and finding an AED that is out of service may be directed to a nearby docking station having an AED that is in service.

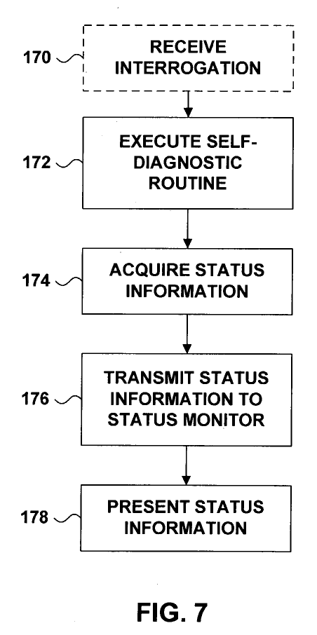

FIG. 7 is a flow diagram illustrating operation of an emergency medical device such as an AED in a system such as system 60. Upon receipt of an interrogation (170) from a status monitor or a docking station, the AED executes a self-diagnostic routine (172) and acquires status information from the self-diagnostic routine (174). The interrogation (170) is optional, and the AED may execute a self-diagnostic routine (172) and acquire status information (174) without an interrogation. The AED may execute a self-diagnostic routine (172) on a periodic basis, for example, or in response to a detected problem in the AED. In some circumstances, the AED may execute a self-diagnostic routine (172) in response to a detected problem in the docking station.

The AED may communicate the status information to a remote unit such as a status monitor (176). The AED may communicate the information to the status monitor directly, or indirectly, e.g., via the docking station. The AED may also present status information locally (178) via one or more output elements.

As noted above, a docking station or an AED may acquire and transmit status information to a remote unit such as a status monitor in response to an interrogation, or may do so without an interrogation. In a typical implementation, it is desirable that the docking station, AED and status monitor have current, consistent status information. In the event of a change in the status by an AED, for example, it is desirable that the docking station associated with the AED and the status monitor receive the AED status information promptly. In this way, the docking station and the status monitor may promptly reflect the true status of the AED. A responsible person may observe the same status information from the status monitor, from the docking station, or from the AED. The status monitor may also follow up with an interrogation to assure that the change in status is not a false alarm.

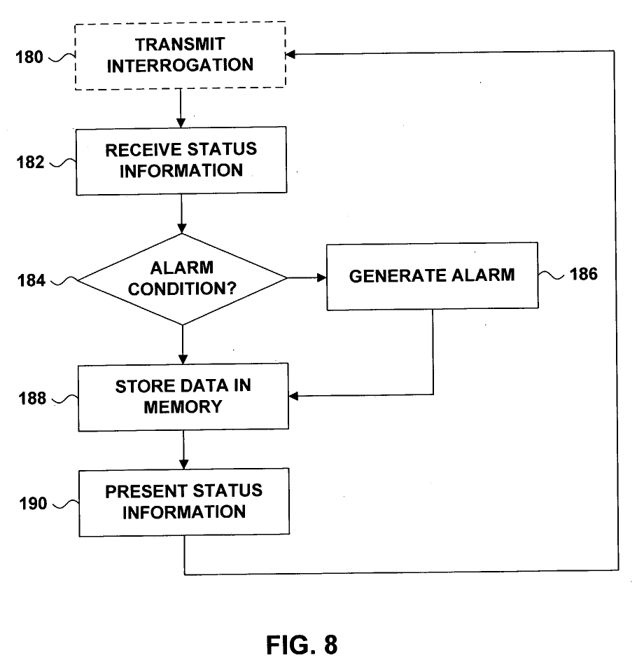

FIG. 8 is a flow diagram illustrating operation of a status monitor in a system such as system 60. The status monitor may transmit one or more interrogations to one or more AEDs or one or more docking stations in the system (180) and may receive status information in response (182). The interrogation (180) is optional, and the AEDs or docking stations may transmit status information without an interrogation, for reasons such as those described above.

The received status information may include an alarm condition. An alarm condition may be a function of the status information, e.g., any status information that reflects a problem or other need for prompt attention. When the received status information includes an alarm condition (184), the status monitor may generate an alarm (186) to alert a responsible person to the need for prompt attention. The alarm may include status information received from an AED or a docking station that may indicate the nature of the problem.

An alarm condition may also exist when the received status information is routine and indicative of no problems. For example, an alarm condition may exist when the expiration date lapses for batteries in a particular AED. The status monitor may generate an alarm (186) to alert a responsible person to the need to replace the batteries in that AED.

Status information, including alarm conditions, may be stored in memory (188). In this way, the status monitor helps the responsible person maintain a status log showing the status and repair history of the devices in the system. The status monitor may also present the status information so that the responsible person may have access to at least some of the status information pertaining to the devices in the system. The presented status information may include, for example, a summary of the device's location and operational status, such as “ready,” “out of service,” “door open” or “in use.”

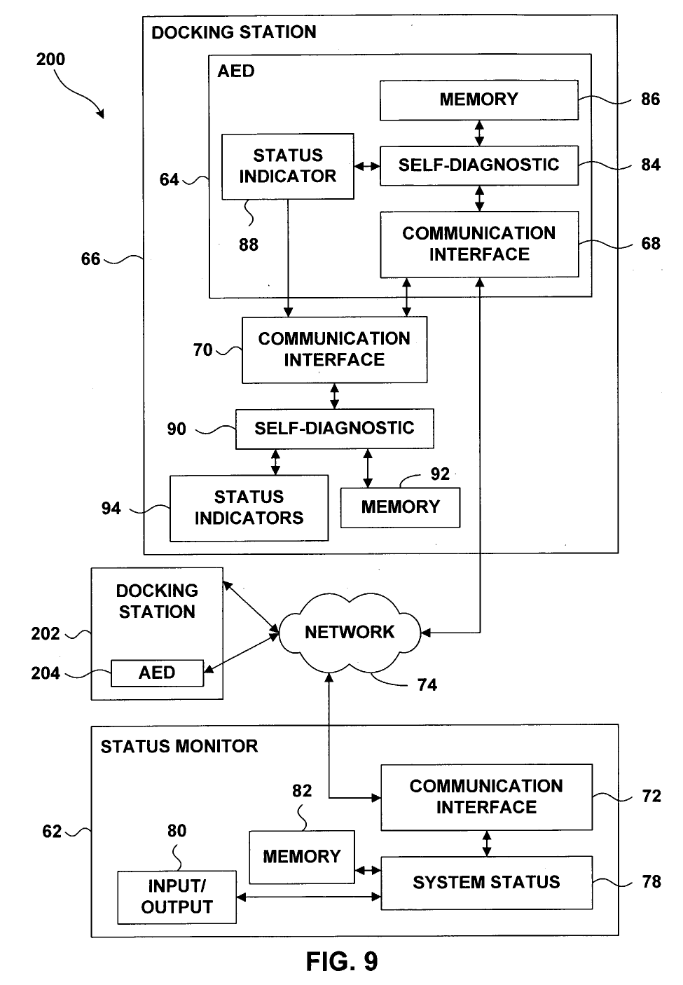

The techniques depicted in FIGS. 4-8 are not necessarily limited to devices in system 60 shown in FIG. 3, but may be adapted to other systems as well. FIG. 9 is a block diagram illustrating another example system 200, in which status monitor 62 receives status information from a plurality of emergency medical devices and docking stations via network 74. System 200 is similar to system 60 in many respects. In system 200, however, AED 64 communicates with status monitor 62 directly, rather than via docking station 66. AED communication interface 68 is coupled to network 74 and to docking station communication interface 70. In this arrangement, docking station 66 communicates with status monitor 62 via AED 64.

FIG. 9 also illustrates that a system may include two-way communication and one-way communication. For example, communication interface 70 of docking station 66 may receive one-way communication from status indicator 88 in AED 64. One-way communication may include, for instance, sensing whether an annunciator on AED 64 is illuminated or not.

FIG. 9 also illustrates how a docking station 202 and an associated AED 204 may communicate with status monitor 62 directly, with neither docking station 202 nor AED 204 serving as a communication intermediary. Docking station 202 and AED 204 may communicate with one another via communication interfaces (not shown). In system 200, status monitor 62 may communicate with any number of docking stations and AEDs, directly or via one or more communication intermediaries.

Advantageously, the invention is not limited to any particular system. Rather, the invention may be practiced with systems of limitless configurations. Any number of docking stations and AEDs may be tracked and monitored with the invention.

Moreover, the invention is not limited to docking stations and AEDs, but may include any emergency medical device. For example, the invention may be practiced with a portable stroke apparatus, or a chest compression device, or a first aid kit, or other medical device. The invention may be practiced with an external defibrillator that is not an AED. A docking station may be associated with any of these medical devices. The docking stations themselves may include any assortment of cabinets, chests, brackets, clasps, bins, closets, kiosks, pedestals and other retaining devices that may be associated with one or more medical devices. In some embodiments, a single docking station may be associated with two or more medical devices. The invention may be practiced in a system that includes a variety of docking stations and a variety of medical devices.

The invention may also be practiced with any number of networks. The invention may be integrated into an existing security network or a private building maintenance network, for example. The invention does not require that all communication links be two-way links. The invention may be practiced with any combination of communication links. In some embodiments, the paths of communication may be restricted, e.g., to prevent misuse or inadvertent or improper programming of a medical device in the system.

The invention may provide other advantages as well. A responsible person may easily monitor the status of any number of medical devices deployed throughout a venue. Presentation of medical device status information and docking station status information at a status monitor may simplify inspection. A responsible person may, at a glance, determine whether any devices in the system are out of service or in need of attention. Record keeping operations, such as maintenance of a status log, are also simplified by a networked system.

Inspection operations may be further simplified by the use of medical device status output elements on a docking station. The output elements of the docking station may be larger, brighter or otherwise more easily accessible than the output elements on the medical device itself. Observation of the output elements of an AED, for example, may require close inspection, while observation of the output elements on the docking station may be perceived from a greater distance or from a wider angle of view. Thus, a responsible person performing routine visual checks may readily obtain status information about the devices in the system without a need for a time-consuming close inspection.

In the event of a problem with any device in the system, the invention facilitates prompt notification of the responsible person. The output elements on the medical devices and the docking stations may alert the responsible person to the problem, and the status monitor may also alert the responsible person to the problem. A remote alert at a monitoring site and a local alert at the site of the device cooperate to improve the likelihood that the problem will be noticed and addressed. In addition, some embodiments of the invention provide for interrogation of a medical device or a docking station, prompting the device to execute a self-diagnostic routine that may discover a problem not previously observed.

In an emergency, the invention may utilize status information to assist with handling the emergency. When an operator retrieves a medical device such as an AED from a docking station, for example, the docking station may immediately communicate that fact to the status monitor. A responsible person may promptly dispatch security or emergency personnel to the general area in which the personnel may be needed. The docking station may also issue an audible alarm that may summon security or emergency personnel to the general site of the emergency. The invention may also advantageously supply status information to docking stations that may assist in an emergency, such as the location of the nearest medical devices that are in service.

Various embodiments of the invention have been described. These specific embodiments are illustrative of the practice of the invention. Various modifications may be made without departing from the scope of the claims. For example, the invention is not limited to AEDs and docking stations, but may be practiced with a variety of medical devices. There may be advantages to deploying the medical devices with docking stations, e.g., to deter mischief and to handle energy consuming operations such as communications. The invention is not limited, however, to medical devices that are associated with docking stations. The invention may encompass, for example, a networked set of emergency medical devices, some of which are associated with no docking station.

The invention is not limited to systems in which medical devices or docking stations are deployed in fixed locations. In some instances, it may be beneficial to deploy a docking station in a mobile platform, such as an ambulance or a vehicle used by a security guard. Moreover, the invention includes embodiments in which a remote unit such as a status monitor is mobile.

Many examples of communication techniques are described for communication among medical devices, docking stations and a status monitor. The invention is not limited to the techniques explicitly described. Communication may be based upon optical communication links, magnetic communication links, infrared communication links, or visual status change detectors. Furthermore, several radio frequency communication links have been described, but the invention is not limited to the techniques explicitly described. A cellular telephone link, for example, may employ any recognized communication protocol, such as code division multiple access (CDMA), Global System for Mobile Communications (GSM), or General Packet Radio Service (GPRS).

Furthermore, the above description is not intended to describe the exclusive functionality of the devices. For example, a docking station or a medical device such as an AED may, for example, maintain a status log separate from the status log, if any, maintained by the status monitor. A docking station may additionally serve as a recharging station in which a medical device may recharge on-board batteries.

Moreover, the invention includes software to carry out the techniques described herein. The invention may be embodied as a computer-readable medium that includes instructions for causing a programmable processor to carry out the methods described above. A “computer-readable medium” includes but is not limited to read-only memory, Flash memory and a magnetic or optical storage medium. The instructions may be implemented as one or more software modules, which may be executed by themselves or in combination with other software.

The instructions and the media are not necessarily associated with any particular computer or other apparatus, but may be carried out by various general-purpose or specialized machines. The instructions may be distributed among two or more media and may be executed by two or more machines. The machines may be coupled to one another directly, or may be coupled through a network.

The invention may also be embodied as one or more devices that include logic circuitry to carry out the functions or methods as described above. The logic circuitry may include a processor that may be programmable for a general purpose or may be dedicated, such as microcontroller, a microprocessor, a Digital Signal Processor (DSP), Application Specific Integrated Circuit (ASIC), and the like. These and other embodiments are within the scope of the following claims.

1. A method comprising:
acquiring first status information from a self-diagnostic routine;
acquiring second status information from an emergency medical device; and
communicating the first status information and the second status information to a remote unit.

2. The method of claim 1, further comprising presenting at least a portion of the first status information and the second status information to a person.

3. The method of claim 1, in which acquiring the second status information comprises:
submitting an interrogation to the medical device; and
acquiring the second status information in response to the interrogation.

4. The method of claim 3, in which acquiring the second status information further comprises receiving the interrogation from the remote unit before submitting the interrogation to the medical device.

5. The method of claim 1, in which acquiring the second status information comprises receiving the second status information from the medical device in accordance with one or more wireless communication protocols.

6. The method of claim 5, in which the wireless communication protocols comprise at least one of Bluetooth, IEEE 802.11a, IEEE 802.11b, IEEE 802.11g, and the HomeRF standard.

7. The method of claim 1, in which communicating the first status information and the second status information to the remote unit comprises establishing communication with the remote unit via a communication network.

8. The method of claim 7, in which the communication network comprises at least one of an alarm network, a telephone network, a local area network, a cellular network, a wireless network, a wide area network, a global computer network and an integrated services digital network.

9. The method of claim 1, in which the medical device includes an external defibrillator.

10. The method of claim 1, in which the medical device includes an automated external defibrillator.

11. A method comprising:
receiving an interrogation for emergency medical device status information;
acquiring the status information from a self-diagnostic routine; and
communicating the status information to a remote unit.

12. The method of claim 1 1, further comprising presenting at least a portion of the status information to a person.

13. The method of claim 11, in which receiving the interrogation for status information comprises receiving the interrogation from the remote unit.

14. The method of claim 11, in which receiving the interrogation for status information comprises receiving the interrogation from a docking station.

15. The method of claim 11, in which communicating the status information to the remote unit comprises establishing communication with the remote unit via a communication network.

16. The method of claim 15, in which the communication network comprises at least one of an alarm network, a telephone network, a local area network, a cellular network, a wireless network, a wide area network, a global computer network and an integrated services digital network.

17. The method of claim 11, in which communicating the status information to the remote unit comprises establishing communication with a docking station in accordance with one or more wireless communication protocols.

18. The method of claim 17, in which the wireless communication protocols comprise at least one of Bluetooth, IEEE 802.11a, IEEE 802.11b, IEEE 802.11g, and the HomeRF standard.

19. The method of claim 11, in which emergency medical device status information comprises at least one of data indicative of a fault, data indicative of low battery power, data indicative of battery failure, and data indicative of the end of a shelf life of a component of the medical device.

20. A method comprising:
receiving emergency medical device status information from a plurality of emergency medical devices; and
presenting at least a portion of the status information to a person.

21. The method of claim 20, further comprising generating an alarm as a function of the status information from at least one of the medical devices.

22. The method of claim 20, further comprising transmitting an interrogation for the status information to the medical devices.

23. The method of claim 20, further comprising transmitting an interrogation for the status information to at least one docking station associated with at least one of the medical devices.

24. The method of claim 20, further comprising:
receiving docking station status information from a docking station associated with at least one of the medical devices; and
presenting at least a portion of the docking station status information to a person.

25. The method of claim 20, further comprising recording the status information in a status log.

26. The method of claim 20, in which emergency medical device status information comprises at least one of data indicative of a fault, data indicative of low battery power, data indicative of battery failure, and data indicative of the end of a shelf life of a component of the medical device.

27. A method comprising:
transmitting an interrogation for status information to a plurality of emergency medical devices; and
receiving the status information from the medical devices in response to the interrogation.

28. The method of claim 27, further comprising generating an alarm as a function of the status information from at least one medical device.

29. The method of claim 27, further comprising presenting at least a portion of the received status information to a person.

30. The method of claim 27, further comprising:
transmitting an interrogation for docking station status information to a plurality of docking stations; and
receiving the docking station status information from the docking stations in response to the interrogation for docking station status information.

31. The method of claim 30, further comprising presenting at least a portion of the docking station status information to a person.

32. The method of claim 27, further comprising recording the status information in a status log.

33. A computer-readable medium comprising instructions for causing a programmable processor to
acquire first status information from a self-diagnostic routine;
acquire second status information from an emergency medical device; and
communicate the first status information and the second status information to a remote unit.

34. The medium of claim 33, the instructions further causing the processor to present at least a portion of the first status information and the second status information to a person.

35. The medium of claim 33, in which the instructions causing the processor to acquire the second status information comprise instructions causing the processor to:
submit an interrogation to the medical device; and
acquire the second status information in response to the interrogation.

36. The medium of claim 35, in which the instructions causing the processor to acquire the second status information comprise instructions causing the processor to receive the interrogation from the remote unit before submitting the interrogation to the medical device.

37. The medium of claim 33, in which the instructions causing the processor to acquire the second status information comprise instructions causing the processor to receive the second status information from the medical device in accordance with one or more wireless communication protocols.

38. The medium of claim 37, in which the wireless communication protocols comprise at least one of Bluetooth, IEEE 802.11a, IEEE 802.11b, IEEE 802.11g, and the HomeRF standard.

39. The medium of claim 33, in which the instructions causing the processor to communicate the first status information and the second status information to the remote unit comprise instructions causing the processor to establish communication with the remote unit via a communication network.

40. The medium of claim 39, in which the communication network comprises at least one of an alarm network, a telephone network, a local area network, a cellular network, a wireless network, a wide area network, a global computer network and an integrated services digital network.

41. The medium of claim 33, in which the medical device is an external defibrillator.

42. The medium of claim 33, in which the medical device is an automated external defibrillator.

43. A computer-readable medium comprising instructions for causing a programmable processor to:
receive an interrogation for emergency medical device status information;
acquire the status information from a self-diagnostic routine; and
communicate the status information to a remote unit.

44. The medium of claim 43, the instructions further causing the processor to present at least a portion of the status information to a person.

45. The medium of claim 43, in which the instructions causing the processor to receive the interrogation for status information comprise instructions causing the processor to receive the interrogation from the remote unit.

46. The medium of claim 43, in which the instructions causing the processor to receive the interrogation for status information comprise instructions causing the processor to receive the interrogation from a docking station.

47. The medium of claim 43, in which the instructions causing the processor to communicate the status information to the remote unit comprise instructions causing the processor to establish communication with the remote unit via a communication network.

48. The medium of claim 47, in which the communication network comprises at least one of an alarm network, a telephone network, a local area network, a cellular network, a wireless network, a wide area network, a global computer network and an integrated services digital network.

49. The medium of claim 43, in which the instructions causing the processor to communicate the status information to the remote unit comprise instructions causing the processor to establish communication with a docking station in accordance with one or more wireless communication protocols.

50. The medium of claim 49, in which the wireless communication protocols comprise at least one of Bluetooth, IEEE 802.11a, IEEE 802.11b, IEEE 802.11g, and the HomeRF standard.

51. The medium of claim 43, in which emergency medical device status information comprises at least one of data indicative of a fault, data indicative of low battery power, data indicative of battery failure, and data indicative of the end of a shelf life of a component of the medical device.

52. A computer-readable medium comprising instructions for causing a programmable processor to:
receive emergency medical device status information from a plurality of emergency medical devices; and
present at least a portion of the status information to a person.

53. The medium of claim 52, the instructions further causing the processor to generate an alarm as a function of the status information from at least one of the medical devices.

54. The medium of claim 52, the instructions further causing the processor to transmit an interrogation for the status information to the medical devices.

55. The medium of claim 52, the instructions further causing the processor to transmit an interrogation for the status information to at least one docking station associated with at least one of the medical devices.

56. The medium of claim 52, the instructions further causing the processor to:
receive docking station status information from a docking station associated with at least one of the medical devices; and
present at least a portion of the docking station status information to a person.

57. The medium of claim 52, the instructions further causing the processor to record the status information in a status log.

58. The medium of claim 52, in which emergency medical device status information comprises at least one of data indicative of a fault, data indicative of low battery power, data indicative of battery failure, and data indicative of the end of a shelf life of a component of the medical device.

59. A computer-readable medium comprising instructions for causing a programmable processor to:
transmit an interrogation for status information to a plurality of emergency medical devices; and
receive the status information from the medical devices in response to the interrogation.

60. The medium of claim 59, the instructions further causing the processor to generate an alarm as a function of the status information from at least one medical device.

61. The medium of claim 59, the instructions further causing the processor to present at least a portion of the received status information to a person.

62. The medium of claim 59, the instructions further causing the processor to:
transmit an interrogation for docking station status information to a plurality of docking stations; and
receive the docking station status information from the docking stations in response to the interrogation for docking station status information.

63. The medium of claim 62, the instructions further causing the processor to present at least a portion of the docking station status information to a person.

64. The medium of claim 59, the instructions further causing the processor to record the status information in a status log.

65. A device comprising:
an electrical source to generate a shock to defibrillate a heart;
at least two electrodes to deliver the shock to the heart;
a processor to execute a self-diagnostic routine and to acquire status information as a function of executing the self-diagnostic routine; and
a communication module to receive an interrogation for the status information and to communicate the status information to a second device.

66. The device of claim 65, in which the device is an automated external defibrillator.

67. The device of claim 65, in which the device is portable.

68. The device of claim 65, in which the communication module is configured to communicate with at least one of a docking station and a network in accordance with one or more wireless communication protocols.

69. The device of claim 68, in which the wireless communication protocols comprise at least one of Bluetooth, IEEE 802.11a, IEEE 802.11b, IEEE 802.11g, and the HomeRF standard.

70. A device comprising:
a processor to execute a self-diagnostic routine and to acquire status information as a function of executing the self-diagnostic routine; and
a communication module to receive an interrogation for the status information and to communicate the status information to a second device,
wherein the device is a portable medical device.

71. The device of claim 70, in which the device is an external defibrillator.

72. The device of claim 70, in which the device is an automated external defibrillator.

73. The device of claim 70, in which the communication module is configured to communicate with at least one of a docking station and a network in accordance with one or more wireless communication protocols.

74. The device of claim 73, in which the wireless communication protocols comprise at least one of Bluetooth, IEEE 802.11a, IEEE 802.11b, IEEE 802.11g, and the HomeRF standard.

75. A device comprising:
a docking element to retain an emergency medical device;
a communication module to receive first status information from the medical device;
a processor to execute a self-diagnostic routine and acquire second status information as a function of executing the self-diagnostic routine; and
an output to present at least a portion of the first status information and at least a portion of the second status information.

76. The device of claim 75, in which the communication module is configured to receive the first status information in accordance with one or more wireless communication protocols.

77. The device of claim 76, in which the wireless communication protocols comprise at least one of Bluetooth, IEEE 802.11a, IEEE 802.11b, IEEE 802.11g, and the HomeRF standard.

78. The device of claim 75, in which the communication module is configured to transmit at least a portion of the first status information and at least a portion of the second status information to a remote unit.

79. The device of claim 75, in which the docking element comprises:
a compartment sized to receive the medical device; and
a door to secure the medical device inside the compartment.

80. A device comprising:
a communication module to receive status information from a plurality of emergency medical devices; and
an output device to present at least a portion of the received status information to an operator.

81. The device of claim 80, in which the communication module is further configured to transmit an interrogation to the medical devices.

82. The device of claim 80, in which the communication module is further configured to receive docking station status information from a plurality of docking stations.

83. The device of claim 80, further comprising memory to record the received status information.

84. The device of claim 80, further comprising a processor to generate an alarm as a function of the received status information.

85. A system comprising:
a plurality of emergency medical devices; and
a remote unit that interrogates at least one medical device in the plurality over a communication network and that receives status information in response to the interrogation.

86. The system of claim 85, in which at least one medical device in the plurality is portable.

87. The system of claim 85, in which at least one medical device in the plurality is an external defibrillator.

88. The system of claim 85, in which at least one medical device in the plurality is an automated external defibrillator.

89. The system of claim 85, in which the communication network comprises at least one of an alarm network, a telephone network, a local area network, a cellular network, a wireless network, a wide area network, a global computer network and an integrated services digital network.

90. The system of claim 85, further comprising a docking station sized to receive at least one medical device in the plurality.

91. The system of claim 90, in which the remote unit is further configured to interrogate the docking station over the communication network and to receive docking station status information in response to the interrogation of the docking station.
